(19) United States
(12) Patent Application Publication
(10) Pub. No.: US 2006/0057691 A1
(43) Pub. Date: Mar. 16, 2006

(54) Process for the extraction of polyhydroxyalkanoates from biomass

(76) Inventors: Karunakaran Narasimhan, West Chester, OH (US); Roland George Severson JR., Cincinnati, OH (US); Susan Eileen Buescher, Cincinnati, OH (US); Angella Christine Cearley, Hamilton, OH (US); Michael Steven Gibson, Cincinnati, OH (US); Stanley James Welling, Liberty Township, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/225,357

(22) Filed: Sep. 13, 2005

Related U.S. Application Data
(60) Provisional application No. 60/610,090, filed on Sep. 15, 2004.

(51) Int. Cl. C12P 7/62

(52) U.S. Cl. 435/135

(57) ABSTRACT

The present invention relates to a process for extracting polyhydroxyalkanoate from a biomass, comprising contacting the biomass with an organic solvent for a time, at a first temperature, and under a pressure that are sufficient to extract polyhydroxyalkanoate from the biomass to provide a composition comprising the organic solvent and polyhydroxyalkanoate; cooling the composition at a second temperature, which is at least about 10° C. lower than the first temperature; and mixing the composition at a second temperature with a power to volume ratio of from about 0.001 KW/m³ to about 100 KW/m³.

### CROSS REFERENCE TO RELATED APPLICATIONS

This application claims the benefit of U.S. Provisional Application No. 60/610,090 filed Sep. 15, 2004.

### FIELD OF THE INVENTION

This invention relates to processes that are useful for the extraction of polyhydroxyalkanoates from a biomass, such as a plant or a bacterial biomass.

### BACKGROUND OF THE INVENTION

Plastics such as polyesters are typically produced from petrochemical sources by well-known synthetic means. These petrochemical-based polymers can take centuries to degrade after disposal. Concern over plastic-waste accumulation in landfills has resulted in a recent movement toward using biodegradable polymers instead.

Bio-based biodegradable polymers, also commonly referred to as “bioplastics,” have not enjoyed great success in the marketplace due to their high production cost. However, advances in biotechnology have led to less expensive methods for their production. In one instance, biodegradable aliphatic copolyesters are now often produced by large-scale bacterial fermentation. Collectively termed polyhydroxyalkanoates, also known as “PHAs,” these polymers can be synthesized by a plant or bacteria fed with a particular substrate, such as glucose, in a fermentation plant. In many instances, the structural or mechanical properties of PHAs can be customized to fit the specifications of the desired end product. PHAs can biodegrade both aerobically and anaerobically.

PHAs are enormously versatile, and as many as 100 different PHA structures have been identified. PHA structures can vary in two ways. First, PHAs can vary according to the structure of the pendant groups, which are typically attached to a carbon atom having (D)-stereochemistry. The pendant groups form the side chain of hydroxyalkanoic acid not contributing to the PHA carbon backbone. Second, PHAs can vary according to the number and types of their repeat units. For example, PHAs can be homopolymers, copolymers, or terpolymers. These variations in PHA structure can cause variations in their physical characteristics. These physical characteristics make PHAs useful for a number of products that may be commercially valuable.

However, in order to have any type of commercially marketable PHA bioplastic product, there is a need for an efficient process for separating such PHAs from the residual biomass.

Numerous solvent-based and other types of extraction techniques are known in the art for extracting PHAs from a biomass. Solvent-based systems (including those utilizing ketones, toluene, alcohols, alone and in combination with other solvents), mechanical systems, and combinations thereof can be used for extracting PHA. However, known solvent-based systems are often inefficient, can cause problematic gelling, aggressive precipitation forming bulk agglomerates, or degradation of particular PHAs. Two-solvent extraction systems are also known, but these systems are often expensive due to the duplicated cost of solvent and can require additional steps when solvent recovery or reuse is sought.

Therefore, there is a need for a more efficient and cost-saving process for extracting the PHA materials from biomass.

### SUMMARY OF THE INVENTION

The present invention relates to processes for extracting polyhydroxyalkanoate from a biomass comprising contacting the biomass with an organic solvent for a time, at a first temperature, and under a pressure that are sufficient to provide a composition comprising the organic solvent and polyhydroxyalkanoate; cooling the composition at a second temperature, which is at least about 10° C. lower than the first temperature; and mixing the composition at the second temperature using a power to volume ratio of from about 0.001 KW/m³ to about 100 KW/m³.

### DETAILED DESCRIPTION OF THE INVENTION

All percentages and ratios used herein are by weight of the total composition and all measurements are made at about 25° C., unless otherwise designated.

The term “PHA” as used herein means polyhydroxyalkanoate.

The invention provides processes for extracting polyhydroxyalkanoate from a biomass, comprising contacting the biomass with an organic solvent for a time, at a first temperature, and under a pressure that are sufficient to provide a composition comprising the organic solvent and polyhydroxyalkanoate; cooling the composition at a second temperature, which is at least about 10° C. lower than the first temperature; and mixing the composition at the second temperature using a power to volume ratio of from about 0.001 KW/m³ to about 100 KW/m³.

In one embodiment, the composition is a solution. In another embodiment, the composition is a suspension. In one embodiment, the composition is a slurry.

In one embodiment, the mixing and cooling are concurrent.

I. Contacting the Biomass with an Organic Solvent

a) Biomass Containing PHA

Polyhydroxyalkanoates can be extracted using the processes of the present invention from sources including, but not limited to, single-celled organisms, such as bacteria or fungi, and higher organisms, such as plants. These sources, together with the PHAs that are biosynthesized from them, are collectively referred to herein as “biomass”. While biomass can comprise wild-type organisms, it also can comprise genetically engineered species specifically designed for the production of particular PHAs of interest. Methods for making such genetically engineered organisms are well known to those skilled in the art.

The biomass useful herein can be substantially dry. As used herein, “substantially dry” means containing less than about 5% water. Substantially dry biomass can be obtained using processes including, but not limited to spray, rotary drum, or freeze drying, before the extraction process is initiated. In one embodiment, a substantially dry biomass contains less than about 2% water; in another embodiment, the biomass contains less than about 1% water, alternatively, the biomass contains no detectable level of water.

Plants useful as biomass organisms include any genetically engineered plant capable of producing PHAs. Such plants include agricultural crops such as cereal grains, oilseeds and tuber plants; other plants include avocado, barley, beet, broad bean, buckwheat, carrot, coconut, copra, corn (maize), cottonseed, gourd, lentil, lima bean, millet, mung bean, oat, oilpalm, pea, peanut, potato, pumpkin, rapeseed (e.g., canola), rice, sorghum, soybean, sugarbeet, sugar cane, sunflower, sweet potato, tobacco, wheat, and yam. Such genetically altered fruit-bearing plants useful in the process of the present invention include, but are not limited to, apple, apricot, banana, cantaloupe, cherry, grape, kumquat, tangerine, tomato, and watermelon. The plants can be genetically engineered to produce PHAs according to the methods disclosed in Poirier, Y., D. E. Dennis, K. Klomparens and C. Somerville, “Polyhydroxybutyrate, a biodegradable thermoplastic, produced in transgenic plants” SCIENCE, Vol. 256, pp. 520-523 (1992); and/or U.S. Pat. No. 5,650,555 to Michigan State University, issued Jul. 22, 1997. In one embodiment, the plants are soybean, potato, corn, or coconut plants that are genetically engineered to produce PHAs; in another embodiment, the plant is soybean.

Bacteria that are useful in the present invention include any genetically engineered bacteria that can produce PHAS, as well as bacteria which naturally produce PHAs. Examples of such bacteria include those disclosed in NOVEL BIODEGRADABLE MICROBIAL POLYMERS, E. A. Dawes, ed., NATO ASI Series, Series E: Applied Sciences—Vol. 186, Kluwer Academic Publishers (1990); U.S. Pat. No. 5,292,860 to Kanegafuchi Kagaku Kogyo Kabushiki Kaisha, issued Mar. 8, 1994. In one embodiment, the bacterium is Alcaligenes eutrophus, Escherichia coli, Protomonas extorquens, Methylobacterium extorquens, Pseudomonas putida, Pseudomonas resinovorans, Pseudomonas oleovorans, Pseudomonas aeruginosa, Pseudomonas syringae, Pseudomonas fluorescens, Sphaerotilus natans, Agrobacterium, Rhodobacter sphaeroides, Actinobacillus, or Azotobacter vinelandii.

In one embodiment, the biomass contains a quantity of PHA that is sufficient to make the extraction process described in the present invention economically desirable. In another embodiment, the amount of PHAs in the biomass is at least about 20% of the total dry weight of the biomass; in another embodiment, at least about 50%; in another embodiment, at least about 60%. In one embodiment, the amount of PHA in the biomass is from about 25% to about 90% of the total dry weight of the biomass.

b) Structurally Flexible PHAs:

One or more types of PHAs can be extracted from the biomass.

In one embodiment, the PHAs of the present invention are those referred to herein as “structurally flexible” PHAs, in that the physical disruption caused by the relatively high co-monomer content or particular pendant group chain length, make them generally more ductile and more difficult to crystallize than PHAs that are characterized by having lower co-monomer content and relatively short pendant groups. Examples of structurally flexible PHAs are disclosed in U.S. Pat. Nos. 5,602,227, RE 36,548, and 6,077,931; and U.S. Pat. Nos. 6,043,063 and 6,087,471.

In one embodiment, the PHAs useful in the present invention have a first repeat unit of the structure: 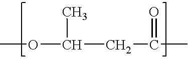
and a second repeat unit having the structure: 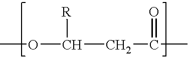
wherein each R is independently a C₃ to C₁₉ alkylene group; and wherein the PHA has from about 75 mol % to about 99 mol % of the first repeat unit, and from about 1 mol % to about 25 mol % of the second repeat unit.

The first and second repeat units can be randomly repeating units. PHAs of the present invention include, for example, random copolymers and block copolymers.

The PHAs of the present methods can have a melt temperature (“Tm”) of from about 80° C. to about 160° C.

In one embodiment, the second repeat unit is hydroxyhexanoate. In one embodiment, the PHA is a hydroxybutyrate-hydroxyhexanote copolymer.

In one embodiment, the PHA is a poly(3-hydroxyalkanoate). In another embodiment, the poly(3-hydroxyalkanoate) is a poly(D-3-hydroxyalkanoate).

In another embodiment, the PHA is a poly(3-hydroxybutyrate)-poly(4-hydroxybutyrate).

In one embodiment, the PHA has from about 3 mol % to about 7 mol % of the second repeat unit. In another embodiment, the PHA has from about 6 mol % to about 12 mol % of the second repeat unit.

The present invention is applicable to PHAs covering a wide range of molecular weights. In one embodiment, the polyhydroxyalkanoate has a molecular weight of from about 100,000 to about 1,500,000. In another embodiment, the PHA has a molecular weight of from about 300,000 to about 800,000.

c) Organic Solvent:

The biomass containing the PHA is combined with an organic solvent to form a composition comprising the organic solvent and PHA.

In one embodiment, the organic solvent is an alcohol, a C₃-C₇ ketone, toluene, ethyl acetate, tetrahydrofuran, acetonitrile, glyme, methyl ester, sulfolane, DMSO, or a combination thereof. Alcohols useful herein include linear or branched alcohols. Exemplary alcohols include methanol, ethanol, n-propanol, isopropanol, n-butanol, isobutanol, isopentanol, sec-pentanol, t-pentanol, or a combination thereof. C₃-C₇ ketones useful herein include acetone, methyl ethyl ketone, diethyl ketone, cyclohexanone, or a combination thereof. In one embodiment, the organic solvent is ethanol.

In one embodiment, the organic solvent is substantially anhydrous. As used here, the term “substantially anhydrous” means comprising less than about 1% water; in another embodiment, comprising less than about 0.5% water; in another embodiment, comprising less than 0.1% water. In one embodiment, the organic solvent is substantially anhydrous ethanol.

In one embodiment, the organic solvent is a solvent in which PHA is substantially insoluble at a temperature of up to about 50° C. As used herein, the term “substantially insoluble” means that no more than about 1% of PHA is soluble by weight; in another embodiment, no more than 0.5% PHA is soluble by weight; in another embodiment, no more than 0.1% PHA is soluble by weight.

In another embodiment, the organic solvent is a solvent in which PHA is substantially soluble at a temperature of up to about 50° C. As used herein the term “substantially soluble” means that at least about 90% of the PHA is soluble by weight; in another embodiment, at least about 95% of the PHA is soluble by weight; in another embodiment, at least about 98% of the PHA is soluble by weight.

In one embodiment, the invention provides an efficient and low-cost PHA extraction process by using a single organic solvent in which PHA is substantially insoluble at a temperature of up to about 50° C., or a combination of an organic solvent in which PHA is substantially soluble at a temperature of up to about 50° C. and an organic solvent in which PHA is substantially insoluble at a temperature of up to about 50° C. In this embodiment, at moderate temperatures and/or pressures and a lower residence time than other extraction processes known in the art can be used.

In one embodiment, the organic solvent is a solvent in which PHA is substantially insoluble. The solvency characteristics of the organic solvent can be modified by changing the temperature and/or pressure and dissolving some or all of the PHA at moderately higher temperature and pressure. Applicants believe that using an organic solvent in which PHA is substantially insoluble at ambient temperature and pressure can provide the benefits of using a single solvent system, including solubilizing some or all of the PHA at moderate temperature and pressure, and providing significant precipitation and yield. Using an organic solvent in which PHA is substantially insoluble at a temperature of up to about 50° C. can provide certain advantages, including the use of a single main solvent both for dissolution and precipitation. Using an organic solvent in which PHA is substantially insoluble at a temperature of up to about 50° C. can allow higher loading of the PHA into the organic solvent with a decreased residence time, which can minimize molecular weight reduction of the PHA. Using an organic solvent in which PHA is substantially insoluble at a temperature of up to about 50° C. can also enable more efficient separation of the polymer. Using an organic solvent in which PHA is substantially insoluble at a temperature of up to about 50° C. can minimize or eliminate the requirement for another solvent for washing. This system can be feasible for batch or semi-continuous processes, and can provide an economic advantage to the extraction process due to, for example, a reduction in solvent inventory, the number and amount of solvents used, and the type of solvents used.

In another embodiment, the organic solvent is a solvent in which PHA is substantially soluble at a temperature of up to about 50° C., optionally including combining the organic solvent with an organic solvent in which PHA is substantially insoluble at a temperature of up to about 50° C., or pressure separation of the PHA. A solvent in which PHA is substantially soluble at a temperature of up to about 50° C. can solubilize the PHA at moderate temperature, with or without pressure that is above ambient pressure. One of skill in the art will recognize that the benefits of the enhanced solubility at a temperature of up to about 50° C., however, might, in some instances, be balanced with a need for pressure filtration of the dissolved PHA or the use of, for example, water or an organic solvent in which PHA is substantially insoluble at a temperature of up to about 50° C. in order to precipitate the PHA. Without wishing to be bound by theory, Applicants believe that pressure filtration and/or the use of a second solvent can enhance yield of the recovered PHA or minimize gelling of the PHA. Applicants believe that using an organic solvent in which PHA is substantially soluble at a temperature of up to about 50° C. can provide several advantages including, higher loading of PHA into the organic solvent, and simplifying the dissolution and biomass filtration. In addition, the molecular weight of the PHA is typically more stable, and less prone to reduction, when using an organic solvent in which PHA is substantially soluble at a temperature of up to about 50° C.

One of skill in the art will recognize that the selection of organic solvent can be based on a number of factors, including, for an organic solvent in which PHA is substantially insoluble at a temperature of up to about 50° C., enhancing PHA solubility at moderate temperatures and/or pressures, enhancing the precipitation or separation of PHA at lower temperatures, the ability of the organic solvent to serve as a washing agent, the molecular weight stability of the PHA to be extracted, compatibility of the organic solvent with the biomass type (either dry or wet), the volume of the organic solvent required, and the cost of the organic solvent. Selection of a solvent in which PHA is substantially insoluble can also include adjusting the temperature and/or pressure to enhance solubility.

The present methods can be performed using an organic solvent to PHA ratio that is typically lower than that of other extraction systems known in the art. In one embodiment, the ratio of organic solvent to polyhydroxyalkanoate is from about 5 parts to about 100 parts organic solvent to about 1 part polyhydroxyalkanoate by weight. In another embodiment, the ratio of organic solvent to polyhydroxyalkanoate is from about 10 to about 30 parts organic solvent to about 1 part polyhydroxyalkanoate by weight. In another embodiment, the ratio of organic solvent to polyhydroxyalkanoate is from about 15 parts to about 20 parts organic solvent to about 1 part polyhydroxyalkanoate by weight. In another embodiment, the ratio of organic solvent to polydroxyalkanoate is from about 15 parts organic solvent to about 1 part polyhydroxyalkanoate by weight. In one embodiment, the biomass comprises from about 30% to about 90% of PHA by weight, alternatively the biomass comprises about 60% of PHA by weight.

In one embodiment, the biomass comprises less than about 8% water; in another embodiment, less than about 5% water, in another embodiment, less than about 2% water, and in another embodiment, the biomass contains no measurable quantity of water.

d) Contacting Time, First Temperature, and Pressure

In one embodiment, the time is for from about 5 to about 120 minutes. In another embodiment, the time is for from about 5 to about 20 minutes.

The molecular weight of the PHA to be extracted can also impact the selection of contacting time. For example, if a lower molecular weight PHA is desired, the contacting time can be longer because degradation of the PHA in the solvent will be less of a concern. If, however, a higher molecular weight PHA is desired, the contacting time can be shorter to minimize significant degradation of the PHA. For example, the contacting time could be varied between 5 minutes and several hours to obtain both higher molecular weight and lower molecular weight PHA.

The solvent used in the extraction can also impact the selection of contacting time. For example, if the solvent has a free hydroxyl or other reactive group, the contacting time can be shorter to minimize significant degradation of the PHA.

In one embodiment, the first temperature is from about 80° C. to about 130° C. In another embodiment, the first temperature is from about 80° C. to about 120° C. In one embodiment, the first temperature is from about 80° C. to about 100° C. and the organic solvent is a solvent in which PHA is substantially insoluble at ambient temperature and pressure.

In one embodiment, the pressure is from about 1 bar to about 6 bar. The pressure can depend on the first temperature and the boiling point of the organic solvent. For example, for a high boiling solvent such as DMSO, the pressure required to reach the first temperature can be about 1 bar. For a lower boiling solvent such as acetone, the pressure required to reach the first temperature can be about 6 bar.

In another embodiment, the process comprises contacting the biomass with an organic solvent for from about 5 to about 120 minutes, at a first temperature of from about 80° C. to about 130° C., and at a pressure of from about 1 to about 6 bar.

e) Cooling the Composition at a Second Temperature

In one embodiment, the composition is cooled at a second temperature, which is at least about 10° C. lower than the first temperature.

In one embodiment, the second temperature is from about 10° C. lower than the first temperature to about 0° C. In another embodiment, the second temperature is from about 50° C. to about 0° C.

In one embodiment, the cooling occurs for from about one minute to about 24 hours.

Cooling the organic solvent can result in precipitation of the PHA.

The contacting and/or cooling steps provided above can also comprise mixing. Mixing can be performed using methods known to those skilled in the art. For example, the mixing can be performed by using propellers, turbines, screw conveyors, or mixtures thereof. In one embodiment, the mixing is performed by using a plug flow concept with a screw conveyor.

f) Power to Volume Ratio

The composition is mixed at a second temperature with a power to volume ratio of from about 0.001 KW/m³ to about 100 KW/m³. The power to volume ratio can be applied throughout the cooling step, or for any portion thereof.

In one embodiment, the power to volume ratio is from about 0.005 KW/m³ to about 50 KW/m³. In another embodiment, the power to volume ratio is from about 0.001 KW/m³ to about 50 KW/m³. In one embodiment, the power to volume ratio is from about 0.005 KW/m³ to about 100 KW/m³.

Mixing the composition at the power to volume ratio can counteract the tendency of the PHA to precipitate aggressively upon cooling the composition at the second temperature. Mixing the composition at the power to volume ratio can produce a slurry, facilitating extraction. One of skill in the art will recognize that the selection of a power to volume ratio can vary, depending on the solvent or on the PHA.

In one embodiment, the power to volume ratio is from about 0.005 KW/m³ to about 100 KW/m³. In this embodiment, the polyhydroxyalkanoate has from about 3 mol % to about 7 mol % of the second repeat unit; or the second repeat unit is 3-hydroxyhexanoate; or the second repeat unit is D-3-hydroxyhexanoate.

In one embodiment, the power to volume ratio is from about 0.001 KW/m³ to about 50 KW/m³. In this embodiment, the polyhydroxyalkanoate has from about 6 mol % to about 12 mol % of the second repeat unit; or the second repeat unit is hydroxyhexanoate; or the second repeat unit is D-3-hydroxyhexanoate.

In one embodiment, the power to volume ratio is from about 0.005 KW/m³ to about 100 KW/m³ and the PHA comprises from about 3 mol % to about 7 mol % of hydroxyhexanoate repeat units. In another embodiment, the power to volume ratio is from about 0.001 KW/m³ to about 50 KW/m³ and the PHA comprises from about 6 mol % to about 12 mol % of hydroxyhexanoate repeat units.

In one embodiment, the mixing occurs for from about 10 minutes to about 24 hours.

The mixing can be any efficient mechanical mixing or handling. In one embodiment, the mixing includes mechanical transport to enhance mobility and to manage precipitation during the cooling process.

In one embodiment, the process is a continuous process. In another embodiment, on-line mechanical mixing is used. In one embodiment, a plug flow concept including a mechanical transportation system such as a screw conveyor is used to provide the power to volume ratio.

In one embodiment, the mixing can be varied to produce different morphology of the precipitated PHA. For example, particles and flakes can be produced using a screw conveyor or mixing ribbons. In another example, flakes and sheets can be produced using a rotating drum or belt with doctor blades. Fine powder can be produced using mixing agitators.

In one embodiment, the process is a batch process. The power to volume ratio can be varied to produce varied PHA particle sizes during precipitation.

II. Separation

In one embodiment, the processes further comprise separating the polyhydroxyalkanoate from the organic solvent subsequent to mixing the composition at the above-described power to volume ratio.

In one embodiment, the separating occurs at a temperature of from about 80° C. to about 120° C. In another embodiment, the separating occurs at a temperature of from about 70° C. to about 90° C. In another embodiment, the separating occurs at a temperature that is from about 50° C. to about 70° C.

Separating the PHA from the organic solvent can include filtration, centrifugation, or a combination thereof. In one embodiment, the filtration is performed at a temperature of from about 40° C. to about 90° C. In one embodiment, the filtration is performed at a temperature of from about 45° C. to about 70° C.; alternatively at about 80° C. In one embodiment, the centrifugation is performed at a temperature of from about 40° C. to about 90° C.

III. Precipitation

In one embodiment, the separating comprises precipitating the PHA from the organic solvent. In one embodiment, the precipitating comprises cooling, flashing, or a combination thereof. In another embodiment, the precipitating comprises cooling. In one embodiment, the precipitating allows removal of dissolved impurities.

In one embodiment, the precipitating comprises admixing the organic solvent with water or an organic solvent in which PHA is substantially insoluble at a temperature below about 50° C. In another embodiment, the precipitating comprises admixing water or an organic solvent in which PHA is substantially insoluble at a temperature below about 50° C. to the organic solvent. In one embodiment, the admixing occurs using propellers, turbines, homogenizers, layers of water coated sheets, moving belts, high shear mixers, or a combination thereof. In one embodiment, a tip speed can be selected to obtain the desired product morphology.

In one embodiment, the precipitating comprises cooling the organic solvent to a temperature of from about 20° C. to about 45° C.

IV. Isolation

In one embodiment, the processes further comprise isolating the precipitated PHA. Filtration can be used to recover the precipitated PHA.

In addition to filtration, the isolated PHA can be squeezed and/or placed under pressure in order to remove any remaining organic solvent.

In addition to filtration and/or other recovery methods, isolated PHA can then be washed with a solvent selected from ketones such as acetone, methyl ethyl ketone, alcohols such as methanol, ethanol, hydrocarbons such as hexane, heptane, or a combination thereof.

In another embodiment, the choice of the organic solvent can obviate the need for a washing step. For example, impurities can stay dissolved in a solvent such as ethanol or methanol.

In one embodiment, the process comprises contacting the biomass with an organic solvent for from about 5 to about 120 minutes, at a first temperature of from about 80° C. to about 130° C. to provide a composition comprising the organic solvent and polyhydroxyalkanoate; cooling the composition to a second temperature that is at least about 10° C. lower than the first temperature; mixing the composition at a second temperature with a power to volume ratio of from about 0.001 KW/m³ to about 100 KW/m³; precipitating the polyhydroxyalkanoate from the organic solvent; and isolating the precipitated polyhydroxyalkanoate.

V. Drying

In one embodiment, the isolated PHA can be dried using well-known methods to remove remaining organic solvent.

VI. Recycling of Solvent

After the step of isolating the PHA, in one embodiment, the organic solvent can be recovered and recycled and/or re-used by well known methods.

VII. Other Process Parameters

In one embodiment, depending on the type of morphology (flake, fiber, powder, film) desired in the precipitated PHA, process parameters can be altered to obtain such morphologies. For example, the method of precipitation can be used as a tool to enable the neat polymers morphology (flake, fiber, powder, film) and enhance the purity of the product.

One of skill will recognize that the optimal range of unit operating conditions or individual devices could vary according to the type of raw biomass.

Therefore, the following examples further describe and demonstrate certain embodiments within the scope of the present invention. The examples are given solely for the purpose of illustration, and are not to be construed as limitations of the present invention since many variations of the present invention are possible without departing from its spirit and scope.

### EXAMPLES

### Example 1

Ethanol Process with Screw Conveyor (Mechanical Transportation) and Cooling System (Pilot Scale)

To 100 kg of dried biomass containing about 60% PHA (polyhydroxybutyrate and hydroxyhexanoate copolymer having about 9 mole % hydroxyhexanoate repeat units) is added 300 kg of ethanol (recycle, wash or fresh ethanol with water content less than 3%) at room temperature to form a homogeneous slurry by mixing in a standard pressure reactor. 600 kg of ethanol are preheated to 130° C. The preheated ethanol and biomass slurry are mixed in a pressure reactor and the temperature of about 90-110° C. is maintained for 15 minutes for extraction of PHA. The spent biomass is filtered by passing the extracted solution through a filter, or spent biomass is separated by use of a centrifuge under pressure. Typically the filtration or separation of spent biomass is performed at 90° C. under pressure, to keep ethanol in liquid form.

The mixture of ethanol and PHA is cooled to about 45° C. to about 70° C. though a screw conveyor with cooling capabilities, and mixed for about five to about 30 minutes at a power to volume ratio of from about 0.001 KW/m³ to about 50 KW/m³. As the PHA precipitates in the heat exchanger or piping system, the screw conveyor maintains the PHA particle morphology in powder or flake form and transports precipitated polymer to the downstream PHA polymer separation unit by filter or centrifuge. Wet PHA cake is dried through a rotary drier under vacuum and 60° C. Production of about 55 kg PHA is expected.

### Example 2

Ethanol Process (Process Scale)

To 100 kg of dried biomass containing about 60% PHA (polyhydroxybutyrate and hydroxyhexanoate copolymer having about 5 mole % hydroxyhexanoate repeat units) is added 300 kg of ethanol (recycle, wash or fresh ethanol with water content less than 3%) at room temperature to form a homogeneous slurry by mixing in a standard pressure reactor. 600 kg of ethanol are preheated to 130° C. The preheated ethanol and biomass slurry are mixed in a pressure reactor and the temperature of about 70-120° C. and pressure of about 3 to about 6 bar is maintained for 15 minutes for extraction of PHA. The spent biomass is filtered by passing the extracted solution through a filter at 70° C. to 100° C. under pressure.

The mixture of ethanol and PHA is cooled to about 45° C. to about 70° C. though a screw conveyor with cooling capabilities, and mixed for about 30 minutes at a power to volume ratio of from about 0.005 KW/m³ to about 100 KW/m³. As the PHA precipitates in the heat exchanger or piping system, the screw conveyor maintains the PHA particle morphology in powder or flake form and transports precipitated polymer to the downstream PHA polymer separation unit by filter or centrifuge. Wet PHA cake is dried through a rotary drier under vacuum and 60° C. Production of about 55 kg PHA is expected.

### Example 3

Ethanol Process with High Shear Mixing During Precipitation (Lab Scale)

8.3 grams of dried biomass (containing about 5.00 grams of PHA—polyhydroxybutyrate and hydroxyhexanoate copolymer having 6.5 mol % hydroxyhexanoate) is mixed with 100 grams of ethanol. The mixture is added to a reactor system capable of handling pressure and filtration capability. The material is rapidly heated to 120° C. and maintained at 120° C. for 15 minutes. The extracted mixture of PHA and ethanol is filtered through 2 micron sintered metal filter at the bottom of the reactor. Precipitation occurs on cooling to about 45° C. to about 70° C., and mixing for 30 minutes at a power to volume ratio of from about 0.001 KW/m³ to about 50 KW/m³ is used to keep the precipitated particles participles dispersed. Ethanol is filtered from the precipitated PHA using a Buchner funnel with #40 Whatman filter paper. Filtered PHA is rinsed with ethanol, spread out in watch glass and allowed to air-dry overnight. PHA yield of about 85% is expected.

Throughout this application, various publications are referenced. The disclosures of these publications in their entireties are hereby incorporated by reference into this application in order to more fully describe the state of the art as known to those skilled therein as of the date of the invention described and claimed herein.

The disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for extracting polyhydroxyalkanoate from a biomass, comprising:
a) contacting the biomass with an organic solvent for a time, at a first temperature, and under a pressure that are sufficient to provide a composition comprising the organic solvent and polyhydroxyalkanoate;
b) cooling the composition at a second temperature, which is at least about 10° C. lower than the first temperature; and
c) mixing the composition at the second temperature with a power to volume ratio of from about 0.001 KW/m³ to about 100 KW/m³.

2. The process of claim 1, wherein the time is from about 5 to about 120 minutes.

3. The process of claim 1, wherein the first temperature is from about 80° C. to about 130° C.

4. The process of claim 1, wherein the pressure is from about 1 bar to about 6 bar.

5. The process of claim 1, wherein the power to volume ratio is from about 0.005 KW/m³ to about 100 KW/m³.

6. The process of claim 1, wherein the power to volume ratio is from about 0.001 KW/m³ to about 50 KW/m³.

7. The process of claim 1, further comprising separating the polyhydroxyalkanoate from the organic solvent subsequent to performing step c).

8. The process of claim 7, wherein the separating comprises precipitating the polyhydroxyalkanoate from the organic solvent.

9. The process of claim 8, further comprising recovering the precipitated polyhydroxyalkanoate.

10. The process of claim 1, wherein the organic solvent is an alcohol, a C₃-C₇ ketone, or a combination thereof.

11. The process of claim 1, wherein the organic solvent is methanol, ethanol, propanol, butanol, pentanol, acetone, methyl ethyl ketone, or a combination thereof.

12. The process of claim 1, wherein the organic solvent is substantially anhydrous ethanol.

13. The process of claim 1, wherein the organic solvent is toluene, ethyl acetate, tetrahydrofuran, acetonitrile, glyme, methyl ester, sulfolane, DMSO, or a combination thereof.

14. The process of claim 1, wherein the ratio of organic solvent to polyhydroxyalkanoate is from about 5 to about 100 parts organic solvent to about 1 part polyhydroxyalkanoate by weight.

15. The process of claim 1, wherein the organic solvent is substantially anhydrous.

16. The process of claim 1, wherein the first temperature is from about 80° C. to about 120° C.

17. The process of claim 1, wherein the contacting occurs for from about 5 to about 20 minutes.

18. The process of claim 1, wherein the mixing comprises using on-line mechanical mixing.

19. The process of claim 20, wherein the mixing comprises using a screw conveyor.

20. The process of claim 7, wherein the separating comprises filtering, centrifuging, or a combination thereof.

21. The process of claim 7, wherein the separating occurs at a temperature of from about 45° C. to about 70° C.

22. The process of claim 1, wherein the second temperature is from about 10° C. lower than the first temperature to about 0° C.

23. The process of claim 8, wherein the precipitating comprises cooling, flashing, or a combination thereof.

24. The process of claim 8, wherein precipitating comprises admixing the organic solvent with water or an organic solvent in which polyhydroxyalkanoate is substantially insoluble at a temperature below about 50° C.

25. The process of claim 24, wherein the admixing comprises adding water or the organic solvent in which polyhydroxyalkanoate is substantially insoluble at a temperature below about 50° C. to the organic solvent.

26. The process of claim 24, wherein the admixing occurs using a propeller, turbine, high shear, layers of water coated sheets, moving belts, or a combination thereof.

27. The process of claim 8, wherein the precipitating comprises cooling the organic solvent to a temperature of from about 20° C. to about 45° C.

28. The process of claim 9, wherein the isolating comprises filtering, centrifuging, or a combination thereof.

29. The process of claim 1, wherein the polyhydroxyalkanoate is a hydroxybutyrate-hydroxyhexanoate copolymer with a molecular weight of from about 100,000 to about 1,500,000.

30. The process of claim 1, wherein:
the time is from about 5 to about 120 minutes, the first temperature is from about 80° C. to about 130° C., and the pressure is from about 1 to about 6 bar;
and wherein the polyhydroxyalkanoate has a first repeat unit having the structure: 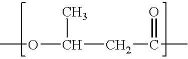
and a second repeat unit having the structure: 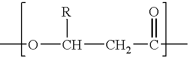
wherein each R is independently a C₂ to C₁₉ alkylene group; wherein the polymer has from about 75 mol % to about 99 mol % of the first repeat unit and from about 1 mol % to about 25 mol % of the second repeat unit;
further comprising:
precipitating the polyhydroxyalkanoate from the organic solvent to provide precipitated polyhydroxyalkanoate; and
recovering the precipitated polyhydroxyalkanoate.

31. The process of claim 30, wherein the power to volume ratio is from about 0.005 KW/m³ to about 100 KW/m³.

32. The process of claim 30, wherein the polymer has from about 6 mol % to about 12 mol % of the second repeat unit.

33. The process of claim 30, The process of claim 42, wherein the polyhydroxyalkanoate is a poly(D-3-hydroxyalkanoate).
